(19) United States
(12) Patent Application Publication
(10) Pub. No.: US 2025/0331712 A1
(43) Pub. Date: Oct. 30, 2025

(54) BINOCULAR VISUAL FIELD EVALUATION THROUGH A CYCLOPEAN NON-EUCLIDEAN FRAMEWORK

(71) Applicant: Gautam Thor, La Jolla, CA (US)

(72) Inventor: Gautam Thor, La Jolla, CA (US)

(21) Appl. No.: 19/263,370

(22) Filed: Jul. 8, 2025

Related U.S. Application Data
(63) Continuation-in-part of application No. 17/699,876, filed on Mar. 21, 2022, now Pat. No. 12,396,635.
(60) Provisional application No. 63/163,511, filed on Mar. 19, 2021.
(60) Provisional application No. 63/182,634, filed on Apr. 30, 2021.
(60) Provisional application No. 63/182,637, filed on Apr. 30, 2021.

(51) Int. Cl. A61B 3/024; A61B 3/00; A61B 3/08; A61B 3/113

(52) U.S. Cl. CPC A61B 3/024; A61B 3/0091; A61B 3/08; A61B 3/113

(57) ABSTRACT

A binocular visual field (VF) testing system and a method employing spherical (non-Euclidean) geometric modeling and eye-tracking technology to generate cyclopean visual field maps are disclosed. The system projects dichoptic stimuli to each eye independently at controlled fixation distances and dynamically records vergence responses using eye tracking. A non-Euclidean spatial encoding model based on logarithmic spirals and Jacobian analysis is applied to the resulting binocular data, enabling three-dimensional topographic field mapping. This approach extends traditional monocular field testing by accounting for retinal curvature, binocular integration, and fixation depth effects, resulting in volumetric visual field models more representative of real-world spatial perception. The system includes hardware (BINOSCOPE) and software modules that implement real-time depth encoding, topological mapping, and boundary detection of peripheral visual fields.

### CROSS REFERENCE TO RELATED APPLICATION

This Applicant is a continuation-in-part of a U.S. patent application Ser. No. 17/699,876 filed on Mar. 21, 2022, which claims priority from U.S. Provisional Patent Appl. No. 63/163,511 filed on Mar. 19, 2021, 63/182,634 filed on Apr. 30, 2021, and 63/182,637 filed on Apr. 30, 2021, all the above applications are incorporated herein by reference in its entirety.

### FIELD OF INVENTION

The present invention relates to a system and method for visual field analysis for screening and monitoring vision disorders, and more particularly, the present invention relates to a dynamic fixation-based system for cyclopean binocular visual field mapping and depth encoding using a non-Euclidean framework.

### BACKGROUND

The term visual field refers to how wide an area an eye can see while focusing on a central point. Visual field assessments are performed to detect scotoma in the vision. Scotoma refers to a blind spot in the field of vision. Scotoma is a degenerated area in the field of vision surrounded by a field of normal vision.

Many diseases of the visual system first manifest as a selective geographic loss of vision at one or more locations. Many brain disorders can also result in blind spots in the vision. Visual field analysis is performed to screen a disease and monitor the progression of the disease during treatment. For example, Glaucoma is a progressive disease in which peripheral vision is lost due to damage in retinal ganglion cells, whose axons form the optic nerve. For example, primary open-angle glaucoma (POAG), which is estimated to affect several million in the United States, can lead to loss of vision if not detected early. Visual field analysis is typically used for detecting, monitoring disease progression, and evaluating new treatments for POAG and other visual disorders.

A visual field analysis also referred to as “perimetry,” involves measuring how well a patient can see at various locations on his retina. Confrontation visual field test is a common way for testing vision field in a clinical setup, wherein a patient, having his one eye covered up or closed, focusses on an object. A doctor can hold up different numbers of fingers in areas of the peripheral visual field, wherein the patient while focusing on the object has to count the number of fingers. Another popular test is a static perimetry test, also performed in a clinical setup. The patient with one eye covered up must look at a center of a bowl-shaped instrument called a perimeter. In the test, small dim lights begin to appear in various places throughout the bowl. While looking at the center target, the patient must report whenever he sees the dim light. Since the lights do not move, the test is referred to as the static perimetry test. Like the static perimetry test but having a moving light target is a Kinetic visual field test.

The visual field test is a subjective examination and depends much on the skills of the person administering the test as well as the understanding of the instructions by the patient. Moreover, the process of testing is tedious and requires the patient to visit a clinic. Going to clinic for visual field assessment can be a problem for home-bound patients. Moreover, the increasing adoption of telemedicine in the medical field mandates the need for newer methods of testing the visual field remotely.

Recently, there have been reports of methods for remotely testing the vison field through a computer screen. For example, Jones, P. R, Portable Perimetry Using Eye-Tracking on a Tablet Computer, A Feasibility Assessment, Trans. Vis. Sci. Tech. 2019; 8(1):17 reported an off-site flat-screen display test for visual field assessment. The known method of visual field assessment using a computer screen suffers from one major limitation, i.e., requiring optimal luminance corrections and/or darkened room.

Also, the traditional visual field-testing systems suffer from one major drawback that is they assess monocular vision in isolation. Traditional perimetry fails to evaluate how both eyes work together in real-world binocular vision.

Thus, a need is appreciated for a system and method for visual field analysis that is devoid of the aforesaid drawbacks of traditional perimetry testing and modern computer-based remote testing.

Herein, the terms “testing”, “assessing”, “analysis”, “assessment”, and interrogation are interchangeably used to broadly refer to visual field analysis for screening any blind spots.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for remotely assessing the visual field of a patient for screening any blind spots or monitoring the progression of any eye defect.

It is another object of the present invention that the system and method can be used to diagnose various eye conditions and brain disorders.

It is still another object of the present invention that the system and method can be used for self-diagnosis without requiring any external help.

It is yet another object of the present invention that the disclosed system and method can be cost-effective to use.

It is a further object of the present invention that the size of the blind spot can be determined.

It is still a further object of the present invention that the disclosed system and method may not require any specific setup and can be performed in ordinary room light.

In one aspect, disclosed is a system and method for assessing visual field of a subject eye of a user for any blind spots. The method includes the steps of presenting a screen that is rendered as a grid, wherein points of the grids are spaced in units of visual angle. A visual focus point is presented on the screen on the screen and a visual stimulus moves on the screen consecutively while the subject eye focuses on the visual focus point and tracks the movement of the visual stimulus. When the visual stimulus enters a blind spot area in the visual field, the movement of the visual stimulus is perceived to be halted but is moving. Upon exiting the blind spot area, the halted visual stimulus is perceived to start moving again. Inputs from the user are taken when the above two events occur. Based on the inputs, the area of the blind spot is mapped.

In one aspect, disclosed is a method for assessing visual field of a subject eye of a user, the method implemented within a system comprising a processor and a memory, the method comprising the steps of presenting a screen, by an interface module implemented within the system and upon processing by the processor, on a display, the screen is rendered as a grid of rows and columns, and cells at the intersection of the rows and the columns, the cells are of square shape, wherein a dimension of each cell is defined in units of visual angle; presenting, by the interface module, a visual focus point on the screen, wherein a location of the visual focus point depends on whether the subject eye is a left eye or a right eye; presenting a visual stimulus on the screen and within a cell of the grid; moving, by an analysis module implemented within the system and upon processing by the processor, the visual stimulus consecutively in a row at a predetermined speed, while the subject eye is focused on the visual focus point; receiving, by the analysis module, a first input from the user, wherein the first input is indicative of a first location of the visual stimulus in the row when the movement of the visual stimulus is perceived by the subject eye to suddenly halt, however the visual stimulus is continuously moving at the predetermined speed; receiving, by the analysis module, a second input from the user, wherein the second input is indicative of a second location of the visual stimulus in the row when the perceived halted visual stimulus is perceived by the subject eye as started moving; moving, by the analysis, the visual stimulus consecutively in a column at the predetermined speed, while the subject eye is focused on the visual focus point; receiving, by the analysis module, a third input from the user, wherein the third input is indicative of a third location of the visual stimulus in the column when the movement of the visual stimulus is perceived by the subject eye to suddenly halt, however the visual stimulus is continuously moving at the predetermined speed; receiving, by the analysis module, a fourth input from the user, wherein the fourth input is indicative of a fourth location of the visual stimulus in the column when the perceived halted visual stimulus is perceived by the subject eye as started moving; and determining, by the analysis module, an area of the blind spot in the subject eye by mapping the first location, the second location, the third location, and the fourth location in the grid relative to the location of the visual focus point, wherein each of the first location, the second location, the third location, and the fourth location are represented in the units of visual angle.

In one implementation of the method, the method further comprises the steps of obtaining, by a registration module implemented within the system and upon processing by the processor, a plurality of properties of a display, the plurality of properties comprises an effective size of the display; and determining, by the registration module, a desired distance range of the subject eye from the display based on the plurality of properties.

In one implementation of the method, the subject eye is the left eye, the visual focus point is positioned on a right side of the screen, the visual stimulus moves from right to left in the row.

In one implementation of the method, the subject eye is the right eye, the visual focus point is positioned on a left side of the screen, the visual stimulus moves from left to right in the row.

In one implementation of the method, the visual stimulus moves cell by cell and row by row from left to right or from right to left, wherein the visual stimulus moves cell by cell and column by column from top to bottom.

In one implementation of the method, the analysis module is configured to cause the visual stimulus to skip one or more cells in a repeated pattern or a predetermined pattern.

In one implementation of the method, the visual stimulus is rendered by highlighting boundaries of the cells, wherein the visual stimulus has a contrasting color relative to a color of a background of the screen.

In one implementation of the method, the visual stimulus moves in the row or the column consecutively leaving a visual trail, wherein the visual trail disappears when the visual stimulus moves to a next row or to a next column.

In one implementation of the method, the method further comprises the steps of determining, by the registration module, a distance of the subject eye from the display; and upon determining the distance, comparing the distance with the desired distance range; and upon comparing, generating first feedback indicating whether the distance is within the desired distance range.

In one implementation of the method, the method further comprises the steps of determining, by the registration module, an undesired movement of the subject eye while the subject eye must be focused on the visual focus point; and upon determining the undesired movement, generating a second feedback indicative of the undesired movement of the subject eye.

In one aspect, disclosed is a visual field (VF) testing system integrating color-segregated stimuli with real-time eye-tracking technology. The system employs dichoptic stimuli, utilizing red-green color filters to segregate visual input, and integrates kinetic campimetry to enhance the accuracy of blind spot mapping.

In one aspect, also disclosed is a visual field (VF) testing system that enables the differentiation between monocular and binocular visual fields based on precise gaze fixation and convergence distances.

In one aspect, disclosed is a method for simultaneous binocular visual field testing which includes real-time mapping of the Cyclopean visual field.

In one aspect, disclosed are a binocular visual field (VF) testing system and method employing spherical (non-Euclidean) geometric modeling and eye-tracking technology to generate cyclopean visual field maps. The system projects dichoptic stimuli to each eye independently at controlled fixation distances and dynamically records vergence responses using eye tracking. A non-Euclidean spatial encoding model based on logarithmic spirals and Jacobian analysis is applied to the resulting binocular data, enabling three-dimensional topographic field mapping. This approach extends traditional monocular field testing by accounting for retinal curvature, binocular integration, and fixation depth effects, resulting in volumetric visual field models more representative of real-world spatial perception. The invention includes hardware (BINOSCOPE) and software modules that implement real-time depth encoding, topological mapping, and boundary detection of peripheral visual fields. It is also envisioned that the system may support future Al-driven machine vision extensions, enabling the VF-based detection of emergent perceptual fields in humans or autonomous visual systems.

In one aspect, the system uses anaglyphic color separation combined with binocular stimulus presentation to map shared and unshared visual field regions without physically occluding either eye. A yellow moving stimulus is displayed on the screen but is perceived differently by each eye due to the anaglyphic glasses: the red-filtered eye sees green, the green-filtered eye sees red. In the binocular overlap zone, the brain fuses these complementary signals, and the stimulus is perceived as yellow, while in the monocular periphery or during blind spot crossings, only a single eye contributes and the stimulus appears red, green, or disappears. The system's controlled campimetry protocol and real-time gaze tracking enable dynamic, precise mapping of this Cyclopean visual field, representing a novel approach to binocular functional assessment.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a block diagram illustrating the architecture and environment of the disclosed system, according to an exemplary embodiment.

FIG. 2 shows a screen of the interface, according to an exemplary embodiment.

FIG. 3 illustrates the mapping of a blind spot using the disclosed system and method, according to an exemplary embodiment of the present invention.

FIG. 4 shows different parameters in the mapping of a blind spot, according to an exemplary embodiment of the present invention.

FIG. 5 illustrates the screen and anaglyph glasses for dichotic stimulus delivery, according to an exemplary embodiment of the present invention.

FIG. 6 shows vergence angles, gaze tracking data, and transformation of stimulus positions into curved coordinate maps using spiral-log encoding, according to an exemplary embodiment of the present invention.

FIG. 7 illustrates the use of chin rest, according to an exemplary embodiment of the present invention.

FIG. 8 shows dichotic stimulus delivery and fixation distance (z), according to an exemplary embodiment of the present invention.

FIG. 9 illustrates Jacobian area mapping, according to an exemplary embodiment of the present invention.

FIG. 10 illustrates toroidal cyclopean visual field map, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an”, and “the” are intended to include the plural forms as well, unless the context indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed are a system and method for analyzing the visual field of a person to screen for blind spots or diagnose any medical condition related to vision and/brain, such as glaucoma. The disclosed system and method permit administering the test remotely, wherein persons in the comfort of their home can do self-diagnosis using the disclosed system and method. No special setup or changing the room lights may be required. For example, the test can be performed in ordinary room lights and may not require dark rooms. Moreover, any existing setup at the home or office of a person can be used, such as a desktop computer with an LCD, a tablet computer, or a laptop can be used. The patients can be saved from traveling to a clinic and waiting for hours for the diagnosis. The disclosed system and method can allow persons to self-diagnose eye defects or monitor existing scotoma/blind spots, at the comfort of their home and at any desired time.

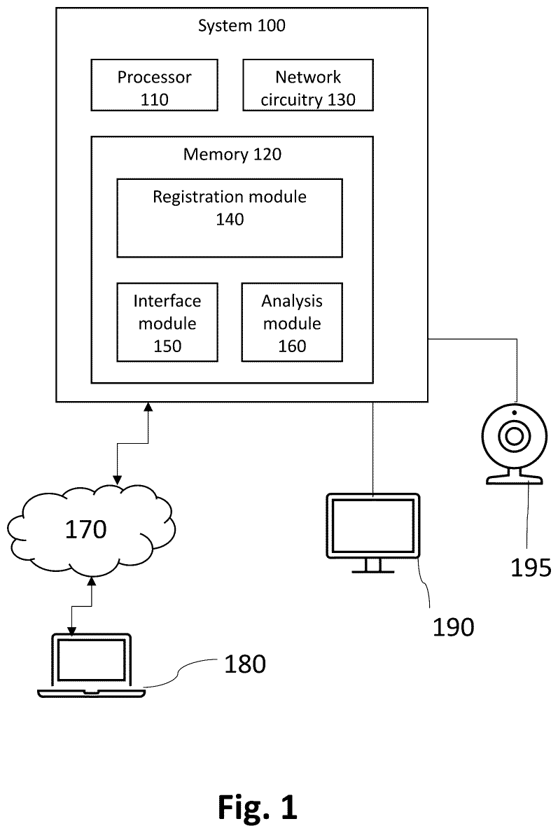

Referring to FIG. 1, which is a block diagram illustrating an exemplary embodiment of the system 100 according to the present invention. The system 100 can include a processor 110 and a memory 120. The processor can be any logic circuitry that responds to, and processes instructions fetched from the memory. The memory may include one or more memory chips capable of storing data and allowing any storage location to be directly accessed by the processor. The system can also include a network circuitry 130 for connecting to a network 170. The network can be wired, wireless network, or a combination of wired and wireless networks. The wired network may include DSL and optical fiber lines. The wireless network may include Bluetooth®, Wi-Fi, WiMAX, and cellular networks including GPRS, LTE, CDMA, 3G, 4G, and 5G. The network can be a secure network, an unsecured network, or a combination of both. The system 100 can connect to one or more remote devices 180 through the network 170. While FIG. 1 shows a single network and a single device, however, it is understood that more than one device can simultaneously connect to the disclosed system through different networks, and a single device can connect with the system through more than one network. For example, a device can connect to the disclosed system through a combination of wired and wireless networks. The device can be a device that includes a display, such as a laptop computer, a desktop computer with a monitor, a tablet computer, and the like. Preferably, the size of the display can be large, for example, more than 15 inches. The device can also include a network circuitry for connecting to the disclosed system. The system can also be connected to a display 190.

As shown in FIG. 1, the memory includes modules according to the present invention for execution by the processor to perform one or more steps of the disclosed methodology. The memory can include a registration module 140, an interface module 150, and an analysis module 160. The registration module, upon execution by the processor, can input details of the user and guide the user to have a posture suitable for the test. The interface module, upon execution by the processor, can present an interface for interacting with the user. The analysis module, upon execution by the processor, can detect blind spots or any other eye/brain disorder. The “user” herein refers to a person using or willing to use the disclosed system and method for visual field assessment. Optionally, application software can be provided on the device. The application software can be developed for different operating systems, such as but not limited to Android™, iOS™, Windows™, Linux, macOS™, or any other known operating platform. In addition to the application software, a website-based interface can also be provided through the World Wide Web. While FIG. 1 shows the system and device as separate objects, however, the system and device can be one. When the system and device are separate, one or more modules of the system or one or more steps of the disclosed methodology can also be implemented on the device, without departing from the scope of the present invention. The system can also be implemented in a server environment, including cloud servers. The server can include more than one server located in one place or geographically scattered at different places. Also, not shown in the drawings, the disclosed system can also connect with other devices, such as a device of a health care professional for sharing the diagnosis or a service provider responsible for storing the medical history of the user.

The registration module can take basic details of the patient through an interface generated by the interface module. The basic details can be optional and can include name, age, gender, and the like. The registration module can also intake the medical history of the user related to the eyes and brain. Also, the details of any previous screening tests for the blind spots taken by the user can be received by the registration module; however, such details are optional. The registration module can detect properties of the display of the device being used by the user. The properties include at least an effective size of the display, resolution of the display, and pixel density. The registration module can also guide the user to position their head relative to the display. The head of the user should be at a predetermined distance from the display. This distance can be calculated by the registration module based on a predefined rule and the detected properties of the display. For example, the optimal distance between an eye and the screen can be about twelve to thirty-six inches. The optimum distance can be absolute or approximate, and deviations in the distance from the absolute optimum distance are within the scope of the present invention. In a preferred embodiment, when the position of the subject eye is at a distance of about twelve inches from the screen, the entire peripheral visual field can be accommodated inside the field of view on a seventeen inches computer display screen and this closer distance setting may be preferable especially when targeting the temporal crest region of the peripheral visual field. Therefore, the optimum distance of the subject eye from the screen may depend upon the effective size of the display.

In certain embodiments, the registration module can also guide the user in positioning their head relative to the screen. Moreover, the registration module can also optionally detect undesired head movements or eye movements during the test. For example, the registration module can use the camera of the device to detect the distance of the head or eyes from the display. Moreover, the camera of the device can be used to detect undesired head movements or eye movements. In one case, the user may be instructed by the registration module to move his head in a predetermined manner to detect the distance of the head from the display. Alternatively, a chin rest with a scale can be provided, and the user can extend the chin head from the display up to the desired distance and can then rest his chin upon the chin rest, thus immobilizing the head. Alternatively, suitable sensors can also be used to detect the distance between the head and the display. For example, a sensor can be coupled to the forehead of the user. The sensors can optionally work with the camera of the display to determine the distance. Any other mechanism to determine the distance of the head from the display is within the scope of the present invention. The chin rest can also include sensors to detect the distance of the chin rest from the display. LIDAR and similar techniques can also be used to determine the distance between the head of the user and the display. Also known are eye-tracking glasses and head mounts that can be used to determine eye movements and optionally measure the distance from the display. Also, a stimulus flash at a known distance from the fixation spot can be provided, wherein the user at the desired distance cannot see the flash, but can see the flash when at the wrong distance. The registration module can monitor the undesired head and eye movements during the test and can provide suitable feedback, such as audio feedback, in case the undesired movements are detected.

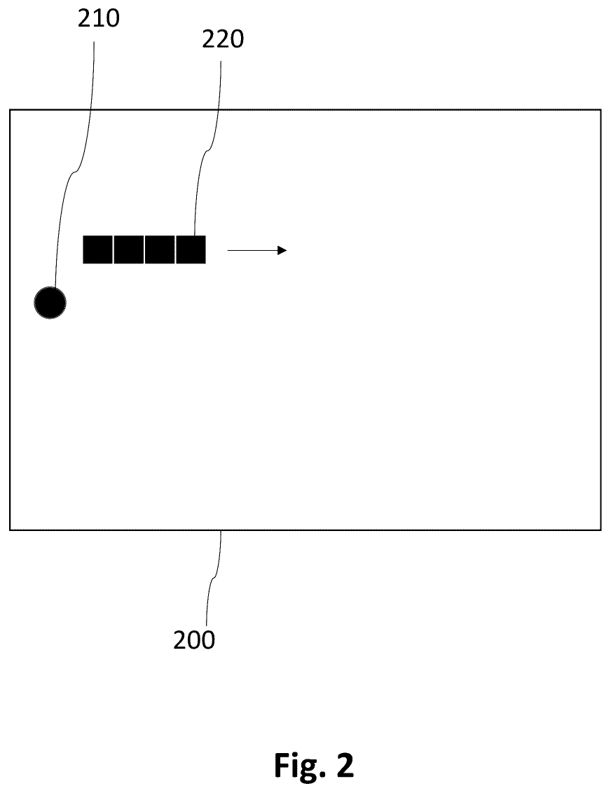

The interface module can provide an interface to receive details from the user as well as to provide instructions and results to the user. Through the interface, the user can interact with the system. The interface can provide a screen implemented on the display for performing the visual field assessment. An embodiment of the screen is shown in FIG. 2. The screen 200 can be virtually divided into a grid having rows and columns, and cells as an intersection of the row and column. However, such a grid may not be visible to the eyes, but the screen can have a plain background. Preferably, the background can be white; however, any color of the background is within the scope of the present invention. Moreover, any pattern as the background is also within the scope of the present invention. A visual stimulus 220 can also be implemented by the interface module on the screen, wherein the visual stimulus can move from left to right or right to left, and from top to bottom of the screen. In certain implementations, the visual stimulus can be a box of a size depending upon the resolution and pixel density of the display. The box can be of a contrasting color compared to the background of the screen. Preferably, the box can be square and black; however, any other shape and contrasting color of the visual stimulus is within the scope of the present invention.

In certain implementations, the visual stimulus can move in rows from left to right or right to left, along with columns from the top to bottom. For a given test, the visual stimulus may, however, move only from left to right or right to left but not both. The visual stimulus can move cell-by-cell and row-by-row from left to right or right to left. Also, the visual stimulus can move cell by cell and column by column from top to bottom. However, one or more cells, or one or more rows, or one or more columns can be skipped by the visual stimulus in a repeated manner or a predetermined pattern without departing from the scope of the present invention. Thus, the visual stimulus can move two cells at a time without departing from the scope of the present invention. Also, it is to be noted that the movement of the visual stimulus vertically from the bottom to the top is also within the scope of the present invention.

In certain implementations, the visual stimulus can move consecutively in a row or column to form a visual line. The visual line starts at the edge or end of a row or column and extends up to the opposite edge or end of the respective row or column. FIG. 2 shows the box shape visual stimulus moving in a contiguous manner, forming a visible trail that starts from the left towards the right, i.e., a visible row/line can be presented by the interface module. However, a previous visual line may disappear when a new visual line begins.

The interface module can also present a visual focus point 210 on screen 200. The visual focus point can also be of a contrasting color relative to the background of the screen. In one case, the visual focus point and the visual stimulus can be of the same color. Alternatively, the visual focus point and the visual stimulus can be of a different color. The visual focus point shown in FIG. 2 is of a solid bullet shape; however, any other size and shape of the visual focus point is within the scope of the present invention. Any animated icon as the visual focus point is also within the scope of the present invention. The position of the visual focus point can depend on the eye being tested. The eye of the user to be tested using the disclosed system is also referred to herein as the subject eye. In one case, when the subject eye is the left eye, the visual focus point can be on the right side of the screen. Similarly, when the subject eye is the right eye, the visual focus point can be on the left side of the screen. FIG. 2 shows the visual focus point on the left of the screen for the right-side subject eye.

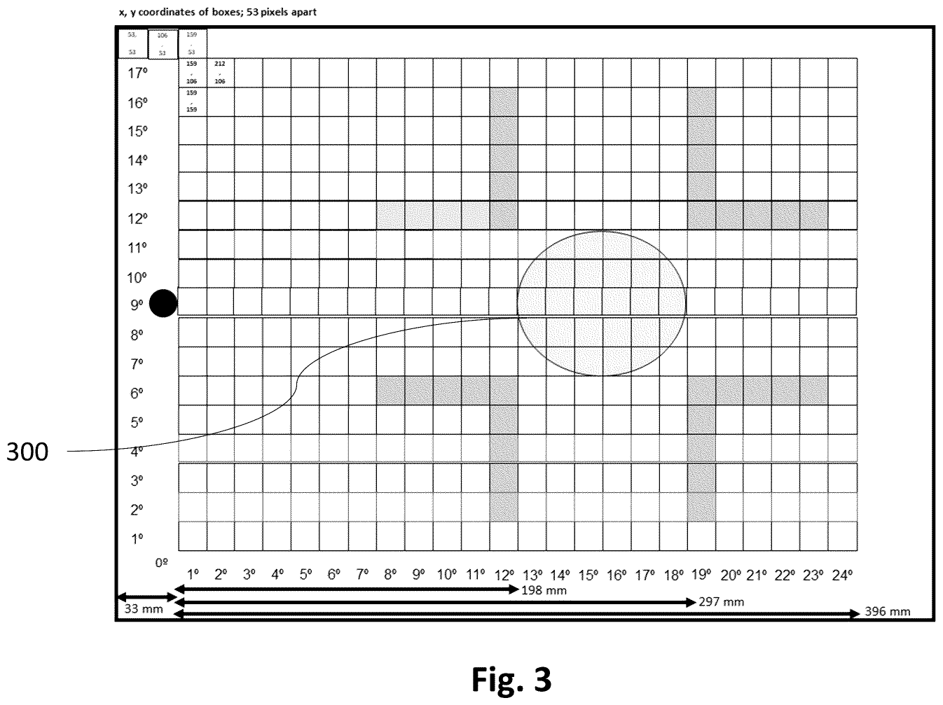

To perform the test, the eye of user other than the subject eye can be covered up. The user can position their head at the desired distance. Thereafter, the user can focus, through the subject eye, on the visual focus point. While keeping the focus on the visual focus point, the analysis module, through the interface module, can cause the visual stimulus to move on the screen in any of the patterns as described above. The visual stimulus can first move horizontally and then vertically, or the visual stimulus can first move vertically and then horizontally. The visual stimulus is visible to the subject eye while being focused on the visual focus point. The subject eye can also perceive the movement of the visual stimulus on the screen. The subject eye, while being focused on the visual focus point, can track the movement of the visual stimulus. However, when the visual stimulus enters a blind spot area of the field of vision of the subject eye, the subject eye perceives that the visual stimulus has stopped moving, which is otherwise moving. The visual stimulus can still be visible to the user, but perceived as stationary. When the visual stimulus exits the blind spot, the visual stimulus can be perceived by the eye as having started to move again. The analysis module can receive input from the user when the moving visual stimulus is perceived by the subject eye to be stopped suddenly. A second input is received from the user by the analysis module when the stopped visual stimulus is perceived by the subject eye to have started moving again suddenly. The input from the user can be taken using any input device coupled to the device of the user, such as a mouse. The user can click to provide the input. Using the two consecutive inputs from horizontal and vertical moving visual stimulus, an area of the blind spot 300 can be mapped by the analysis module, as shown in FIG. 3. In FIG. 3, a full HD monitor of 27 inches (diagonal) is used for the study. The pixel dimensions can be 0.311 mm×0.311 mm, 3.23 pixels per mm (82 pixels per inch). The following Resolution may be used for the Blind-Spot Location Test: 1° of visual angle=1 box=53 pixels=16.5 mm (Stroke (border) of each box to overlap with the stroke of adjacent boxes). The Most Probable Locus of Blind Spot: can be between 12° to 18° along the X-axis and 2° to 3° visual degrees on either side of the equator. For the test, the Right Eye (to be tested) must be perpendicular to the Display surface, and in line with the visual stimulus (53-pixel dia.) on the left edge of the display.

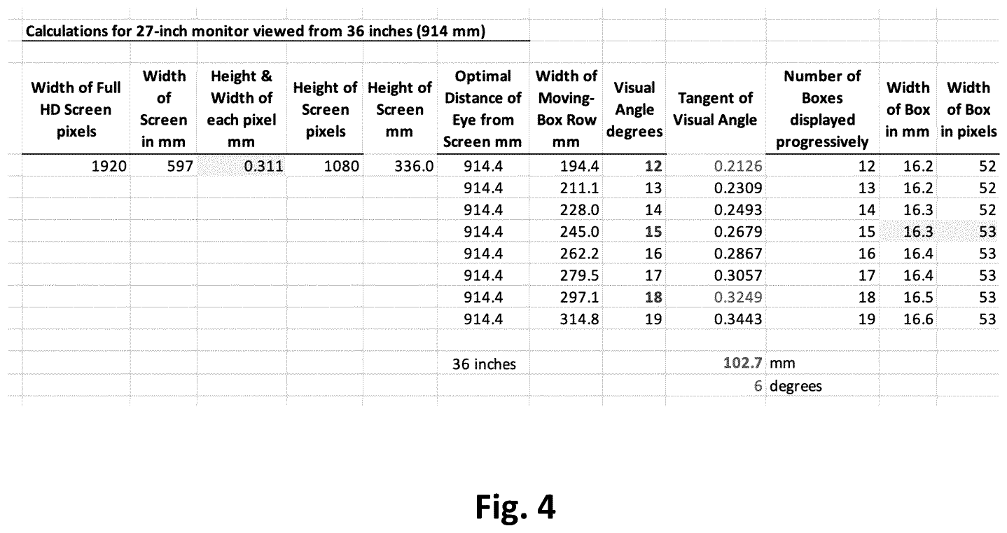

FIG. 4 shows different parameters taken into consideration for mapping the blind spot. The area of the blind spot in the form of a map can be displayed by the analysis module through the interface module. The results can also be shared in different forms, such as PDF, JPG, or any dynamic format, and the results can also be transmitted to other devices, through FTP, HTTP, Email, and the like internet protocols.

The analysis module can also determine the response time of the user, i.e., the difference between the time at which the sudden stop or restarting of the movement of the visual stimulus is perceived by the eye and the time at which the input is received by the analysis module. For example, the analysis module can stop the moving visual stimulus and move the stopped visual stimulus randomly while the subject eye is focused on the visual focus point. The time taken by the user to provide the input can be averaged out to obtain the response time.

Again, referring to FIG. 4, it can be seen that the size of the visual stimulus, such as a blinking square can be proportional to the distance of the user's head from the screen. Optionally, the user can have the freedom to choose the distance or choose the distance from a predefined range. FIG. 4 shows the calculations done for a distance of thirty-six inches i.e., 914 mm. The visual angle can be calculated as the tangent inverse of the length and/or breadth of the screen divided by the distance from the screen.

In certain implementations, the disclosed system and method can be used to diagnose disorders where there is pathology in the retina, such as glaucoma, Brain Tumors, or Retinitis Pigmentosa, and like eye and brain disorders. The disclosed system and method have the advantage that the performance of the tests may not need adjustment for ambient luminosity nor require a darkened room. The test can be displayed on a flat screen (rather than a curved perimetric bowl) with emphasis on the peripheral location of the visual field test points being displayed consistent with the temporal crest projection, outside the “central” region at the fovea onto the nasal retinal regions. This region, representing the peripheral Visual Field after the projected Vision Cone, inverts all 3 axes in image representation derives from the proposed topological model of Visual Field representation. Importantly, the disclosed system and method can be easy to translate to an internet-connected computer screen display module for home testing.

In certain implementations, the visual stimulus can be animated, such as blink. For example, a blinking square can be presented on the screen, wherein the blinking square forms a horizontal line that gradually extends from one side of the screen to the other at 2° per second. The moving and blinking square is normally visible to the subject eye as it extends towards the blind spot. However, as it passes the Blind Spot region, instead of disappearing from the view, the square may seem to halt and will pick up movement once the square passes out of the blind spot. It is anticipated a similar result if the moving stimulus passes a region (including glaucomatous damage)—and importantly, the test point will always be visible. An undetected signal by the user cannot be misinterpreted as a mistake on their part.

In certain implementations, also disclosed are a system and method for binocular visual field mapping based on the realization that the eyes function as curved, spherical surfaces—not planar projections—and that binocular integration in the brain operates through non-Euclidean spatial computation. This system captures visual information using a dichoptic binocular stimulus configuration and transforms it through a spiral-logarithmic encoding model. Unlike conventional approaches, the system dynamically computes field topology based on vergence angle and fixation distance, producing a cyclopean perceptual field map governed by the spherical geometry of the retina. The disclosed system and method for binocular visual field mapping represent the first quantifiable model for binocular visual integration, offering unprecedented accuracy in diagnosing conditions affecting stereoscopic vision, such as amblyopia, strabismus, and neuro-ophthalmic disorders, and subtle binocular suppression or perimetric scotomas.

This results in a first-of-its-kind cyclopean visual field map expressed in three dimensions, derived through binocular data fusion aligned to the geometric structure of the horopter. The system maps corresponding retinal points from each eye and models the cyclopean percept as a toroidal or curved geodesic manifold. In contrast to traditional flat field grids, this technique accounts for both shared and unshared binocular regions and models spatial compression and depth asymptotically via spiral-log convergence. The generated field maps reflect not only where stimuli are seen but also how depth, distance, and periphery are encoded.

The disclosed binocular visual field mapping system is built on a set of non-Euclidean transformations, including:

A spiral-logarithmic depth projection, converting fixation depth Z into the vergence angle θ using:

$$θ = \frac{1}{λ} log ( \frac{Z}{{Z}_{0}} )$$

where Z is the fixation distance, Z₀ is a reference distance, λ is a geometric constant, and θ is the vergence angle.

A Jacobian determinant metric to measure differential area growth:

$$J = \frac{d A}{d Z} = π {θ}^{2}$$

These allow field curvature and surface deformation to be rendered as topological maps in real-time, correlated with eye-tracking data and dichoptic stimulus responses. The field representation is volumetric and vergence-dependent, enabling quantification of both central fixation and peripheral boundary drift across variable depths.

The disclosed binocular visual field mapping system can use a display monitor and anaglyphic 3D glasses for dichoptic stimulation delivery and binocular vision assessment. However, it is to be noted that any other dichoptic stimuli delivery mechanism is within the scope of the present invention. For example, the display may be configured to deliver dichoptic stimuli using wavelength-specific filters, polarized glasses, or split-screen LED presentation to independently stimulate each retina.

Also, the binocular visual field mapping system leverages eye-tracking technology to continuously measure gaze angle changes during binocular stimulus presentation. When both eyes perceive the fused stimulus centrally as yellow, but only detect their respective color components peripherally, the system enables precise binocular disparity mapping. The disclosed binocular visual field mapping system offers a Cyclopean Visual Field Map that may provide novel insights into binocular integration.

The disclosed binocular visual field mapping system includes a binocular field superimposition algorithm that merges monocular field data from each eye into a singular cyclopean projection, delineating central shared visual space and peripheral individual-eye contributions.

The visual field (VF) test is conducted by dynamically moving a dichoptic stimulus on a screen at variable eccentricities, under user-controlled fixation distances. Eye-tracking data are simultaneously captured at high sampling rates to precisely determine the subject's gaze and vergence angle at each instant. The user is instructed to maintain fixation on a target while peripheral stimuli-perceived to be alternating in color and position—are displayed under monocular and binocular conditions. These stimuli traverse through precomputed kinetic paths designed to intersect the peripheral boundary of the VF, allowing for real-time mapping of thresholds.

All positions, timing, and eye-tracking data are pipelined into a computational engine that computes vergence angle “θ” and maps each stimulus location onto a spherical shell representing the retinal surface. The spherical coordinates are then transformed using a logarithmic spiral function of the form:

$$Z ( θ ) = {Z}_{0} \cdot {e}^{λ \cdot Δθ}$$

This transformation permits perceived distance and angular displacement simultaneously, yielding a unified representation of the near, mid, and far-field regions.

A key computational metric employed is the Jacobian determinant:

$$J = \frac{d A}{d Z} = π \cdot {θ}^{2}$$

This measure allows for differential analysis of perceived field expansion, especially in binocular overlap zones, and forms the basis for boundary detection of the Cyclopean Visual Field (CVF).

In the disclosed system, the resultant visual field map is not a 2D surface but rather a 3D toroidal manifold, where individual fields from both eyes are stitched through vergence alignment and angular transformation, thereby generating a Cyclopean Visual Field Map. This map encodes Central vs. peripheral boundaries as a function of fixation depth; Field asymmetries across shared vs. unshared binocular zones; Vergence-induced warping effects consistent with Horopter curvature; and Transition zones modeled as toroidal boundary shifts at critical eccentricities.

The system may also classify patient visual fields using topological invariants, such as edge continuity, field compression ratios, and toroidal asymmetry metrics, thereby transforming VF testing from detection to interpretation

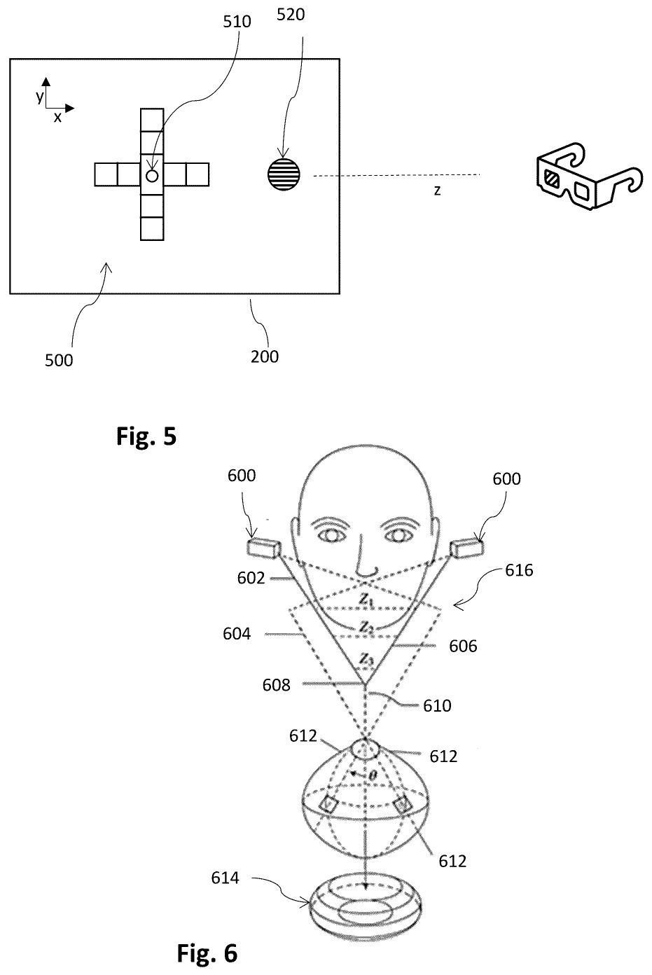

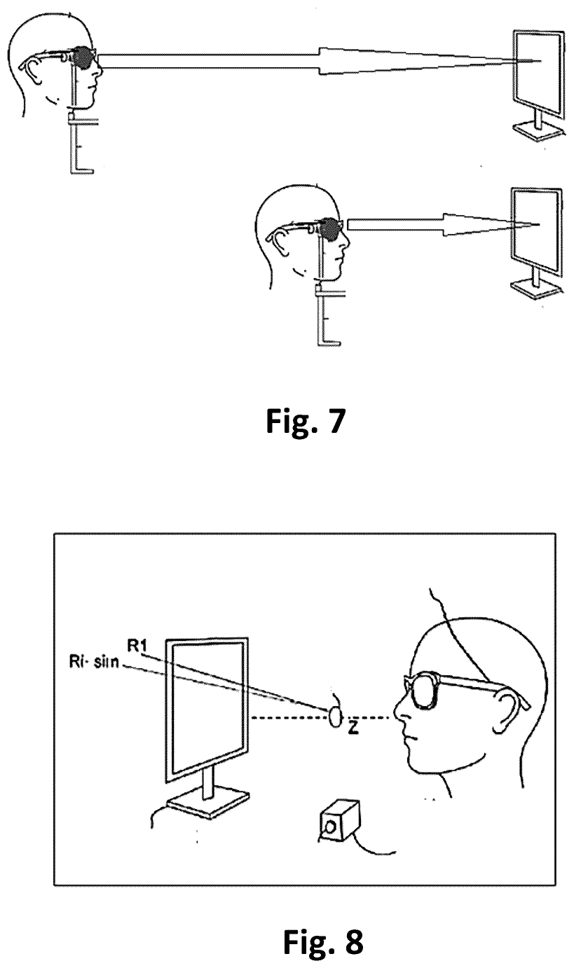

Referring to FIG. 1, the system may also connect to an eye tracking device 195, such as a camera, through which the analysis module can track eye movements of a user. FIG. 5 shows a screen 500 implemented on a monitor 200 implemented by the interface module. The screen may be oriented in the x-y plane while the anaglyph glasses 510 may be oriented, such that the line of sight is along the z-axis. The interface module can deliver a color-segregated (dichoptic) stimulus, i.e., a central yellow target generated on a standard display, while the subject wears red-green anaglyph glasses. For example, the standard display can be an RGB display. When viewed through anaglyphic glasses (with one red and one green lens), each eye perceives only the complementary component of the yellow stimulus. For instance, the eye with a red filter perceives the yellow stimulus as red, while the eye with a green filter perceives it as green.

This enables precise mapping of each eye's contribution to the shared (Cyclopean) field without physically occluding one eye. Also, the dichotic delivery system, according to the present invention, allows driving differential responses from each eye to the same stimulus. Also, this dichoptic delivery enables the system to map which parts of the field are shared or unique to each eye, depending on whether a monocular component is perceived or suppressed at a given location. The dichotic delivery system for binocular field testing is exploited by the system to achieve a form of binocular contrast tagging, without occluding either eye.

The pattern shown on screen 500 consists of a moving cross stimulus in one color channel and a fixation target 520 in the other. The moving stimulus is a small circular spot 510 (yellow on the screen). This yellow disc-shaped moving stimulus moves in a controlled pattern across the visual field. The fixation target is a separate static marker, often a small cross or dot, presented in a different color (commonly green or red), to hold gaze. While the subject wears red/green anaglyphic glasses, the moving stimulus is moved in a predetermined manner to determine eye tracking data. The red-filtered eye blocks red and transmits green→this eye sees the yellow stimulus as green. The green-filtered eye blocks green and transmits red→this eye sees the yellow stimulus as red. In the binocular fusion zone, the brain merges these two monocular signals and perceives the stimulus as yellow again. In shared binocular regions, even though each eye sees only a single-color component, the perceptual result is yellow. In monocular or unshared regions (e.g. peripheral areas or blind spot), the stimulus will appear in only one color, corresponding to the eye that can see it.

The moving bar stimulates regions across the visual field. The moving stimulus (yellow spot) sweeps across the visual field, in a predefined path (horizontal, vertical, or spiral). As it moves, it enters: The central shared (binocular) field→subject perceives yellow, the monocular periphery→subject perceives either red or green, depending on which eye can see that region, and the blind spot of each eye→the stimulus disappears entirely for that eye. If one eye's blind spot is crossed, the stimulus will appear in the complementary color from the other eye only. If the stimulus falls in both blind spots simultaneously, it disappears completely. This can is generated by a software-controlled kinetic campimetry protocol. The moving bar simulates regions across the visual field, while the eye-tracker verifies that the subject maintains fixation on the fixation target. The moving stimulus periodically disappears or reappears based on the subject's blind spot or peripheral boundary, enabling precise mapping.

The eyes of the subject may be tracked by the analysis module using any known technology, such as using cameras. The one or more eye tracking cameras may be used to track eye movements. Such eye tracking cameras may be integrated into the eyeglasses. It is to be noted that any eye tracking technology and method is within the scope of the present invention.

Fixation distance can be varied by physically positioning the test screen at adjustable distances from the subject's eyes, typically ranging from 30 to 60 cm. FIG. 7 illustrates the change in fixation distance. It is to be noted that the distance between the subject and the screen can be varied, thus either or both the screen or the subject can be moved. Thus, the invention provides a controlled, repeatable, and measurable way of adjusting the subject's fixation distance during binocular visual field testing. A novel chinrest with rail guide assembly is also disclosed. The user can stably position the chin over the chin rest. The chin rest is movable on the rail guide or similar mechanism. The raid guide includes a measurement scale, so that the chin rest can be moved by the desired distance towards and away from the screen. Also, the distance between the chin rest and the screen can be measured accurately. This allows for controlling the subject's vergence demand and corresponding fixation distance for each test iteration. Also, the position of the chin rest relative to the rail guide can be electronically determined i.e., distance between the screen and the chin rest can be electronically determined.

Also, the rail guide can be motorized, so that the chin rest can be moved precisely and automatically. This may enable automated adjustment of fixation distance with precise control. This will allow for faster transitions between fixation distances, greater accuracy and reproducibility across tests, and integration of distance changes into the software-controlled testing workflow. The integration of controlled variable fixation distance and gaze angles allows for systematic study of the relationship between fixation distance, gaze angle, and binocular field boundaries.

During each test, the screen remains fixed at a defined depth, and the subject's vergence (convergence angle) is calibrated for that distance using eye-tracking data. Fixation distance is along this Z-axis, and each test session is defined at a single fixed Z value, enabling one to test near, mid, and far vergence positions and compare resulting field maps across depth.

In use, the subject can place their chin in the chin rest at a predefined initial distance, for example, 30 cm. The binocular visual field test is conducted at this initial distance. The chin rest can then be moved to a new calibrated position (e.g., 45 cm, 60 cm, or other target distances), increasing or decreasing the fixation distance. The subject can place their chin again and repeats the test at this new fixation distance. Multiple fixation distances can thus be tested sequentially in a single session, enabling analyses of how binocular field boundaries shift as a function of vergence.

The disclosed system by integrating dichoptic visual stimuli, variable fixation distances, and high-speed binocular eye tracking allows for binocular visual field-testing. All the position, timing, and eye-tracking data are streamed in real time into the analysis module. The invention's novelty lies in its geometrical and functional modeling of binocular integration over a curved retinal surface, which accounts for differential encoding of near and far spatial targets based on fixation-dependent retinal correspondence. The disclosed system further allows for parametric modeling of peripheral field asymmetries as a function of vergence and gaze angle. The platform is modular, supporting future applications in autonomous machine vision, clinical diagnostics, and immersive visual training. The invention is implemented as a testable prototype and validated via normative human data collection and model simulations.

The analysis module uses the spiral log encoding law, which uses logarithmic spirals and Jacobian differentials to transform Euclidean stimulus space into retinal spherical coordinates. The system generates real-time, non-Euclidean field maps and identifies the boundary between shared and unshared visual fields, thus constructing a three-dimensional representation of a cyclopean visual field.

The system using the position, timing, and eye-tracking data may compute the subject's vergence angle θ at each moment based on dual eye gaze vectors; projects the perceived location of each stimulus onto a spherical shell representing the retinal surface and applies a logarithmic spiral transformation:

$$Z ( θ ) = Z 0 \cdot e λ \cdot Δθ Z ( ∖ theta ) = Z\_ 0 ∖ cdot e \hat{} \{ \backslash lambda \backslash cdot \backslash Delta \backslash theta \} Z ( θ ) = Z 0 \cdot e λ \cdot Δθ$$

Wherein Z(θ)Z(\theta)Z(θ) is the transformed radial depth, Z0Z_0Z0 is the baseline reference distance, λ\lambdaλ is the spiral gain constant, Δθ\Delta\thetaΔθ is the angular displacement from the fixation point.

This transformation enables encoding of angular displacement and fixation-dependent depth along a non-Euclidean curved manifold corresponding to the geometry of the eye. Subsequently, a Jacobian determinant is computed for each mapped region:

$$J = dAdZ = π \cdot θ2 J = \backslash frac \{ d A \} \{ d Z \} = ∖ pi ∖ cdot ∖ theta \hat{} 2 J = dZdA = π \cdot θ2$$

where dAdAdA represents the differential projected area on the visual shell, and dZdZdZ the depth interval. This metric supports area-based differential analysis of perceived spatial field expansion-critical for defining and quantifying the Cyclopean Visual Field (CVF) in three dimensions. This enables area-based differential analysis of perceived spatial field expansion, critical for defining the Cyclopean Visual Field (CVF) in 3D space.

System Calibration: The blind spot remains a fiducial marker for calibration, with the new algorithm refining real-time spatial alignment across varying fixation distances.

It is also envisioned that binocular functional assessment, according to the present invention, can enable improved diagnosis and monitoring of binocular dysfunctions, visual fatigue syndromes, and depth perception anomalies.

A method for assessing simultaneous binocular visual fields, wherein a dichoptic stimulus is presented to both eyes using anaglyphic filters, and gaze tracking determines differential fixation responses. A system for mapping the Cyclopean visual field, wherein real-time gaze angles are recorded to compute fused binocular representations of central and peripheral vision. An adjustable fixation mechanism for visual field assessment, wherein a dynamic chin rest enables controlled changes in convergence distance, affecting perimetric boundary shifts. An eye-tracking integrated perimetry system, wherein infrared-based tracking continuously adjusts stimulus presentation in response to real-time gaze shifts.

In certain implementations, also disclosed is a method for encoding and recovering depth information from a three-dimensional scene. The method includes capturing spatial information from the scene using an imaging system that records data on a curved surface. The imaging system may include a single sensor array positioned along a curved, spherical surface, and includes a calibration mechanism using at least one reference point to recover absolute depth scaling. The imaging system may also include two spatially separated curved surfaces simulating binocular vision, and depth calibration is achieved dynamically through convergence of gaze angles between the two surfaces.

Then parameterizing said curved surface as a spherical manifold using a logarithmic spiral coordinate system wherein angular displacement encodes increasing radial depth from the point of observation. Thereafter, mapping each point in three-dimensional space to a corresponding point on the spiral-parameterized spherical surface such that the Z-axis metric is encoded as angular displacement. The mapping may be used to construct a differential geometric representation of depth that does not rely on linear disparity or triangulation.

Thereafter, determining depth from the resulting surface data by applying a transformation that includes calculating the Jacobian matrix of the mapping and extracting its determinant to recover metric depth information from local angular differentials. The logarithmic spiral parameterization may enable one-to-one correspondence between depth in Euclidean space and angular position on the spherical surface.

Referring to FIG. 6, which illustrates the vergence angles, gaze tracking data, and transformation of stimulus positions into curved coordinate maps using spiral-log encoding. FIG. 6 shows two eye-tracking cameras 600, Visual cone 602, fixation distance 604, vergence angle θ 616, color-coded stimulus 606, spiral log encoding 608, Jacobian

$$( \frac{d A}{d Z} )$$

610, retinal shell 612, cyclopean visual field 614.

FIG. 6 shows two eye-tracking cameras 600 for tracking the left and right eye positions. The diagram shows the two eyes converging at a near fixation point. FIG. 6 shows three fixation distances labelled as Z₁, Z₂, and Z₃. Visual cones extend from each eye to common fixation targets. Spiral log encoding curves (logarithmic spirals) are depicted along visual cones on spherical retina shells. Vergence angle θ and resulting Jacobian area differential (dA/dZ) are also marked. FIG. 6 also shows color-coded stimulus panels (red/green dichoptic stimuli) shown at variable fixation points. The resultant cyclopean visual field is shown as a mapped region on an internal toroidal schematic below.

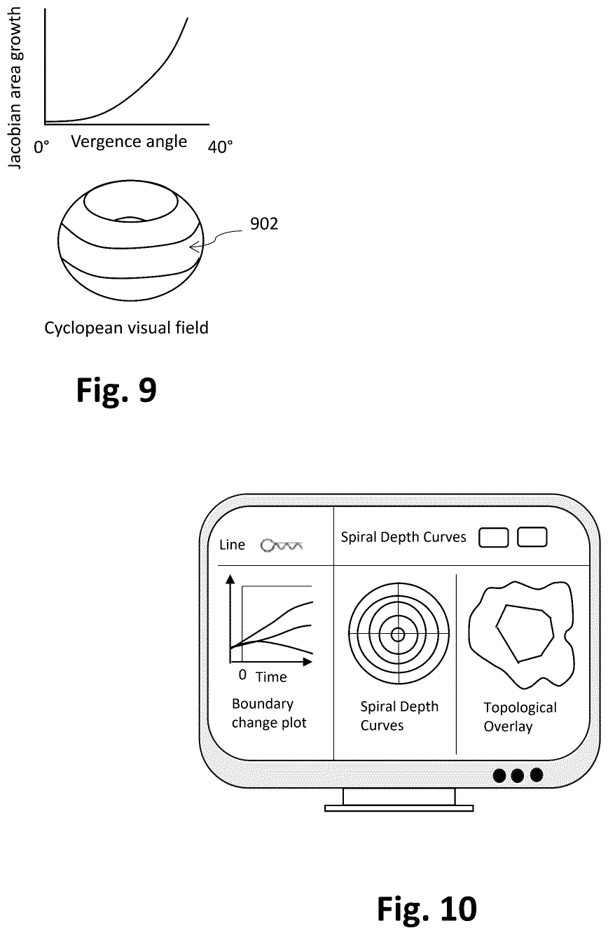

FIGS. 9 and 10 show topological virtual field map simulations, FIG. 9 illustrates comparison of Euclidean vs. non-Euclidean boundary curves at different fixation points, FIG. 10 shows Jacobian derived area differentials and convergence of field limits into a toroidal visual field topology. FIG. 9 shows a Jacobian area mapping graph in which axes are labelled with depth zones and angular divergence. The graph in FIG. 9 depicts area (A) vs. Depth (Z), and the growth curve following J=dA/dZ=πθ². The cyclopean visual field has middle area 900 which is unshared binocular zones. FIG. 10 shows toroidal cyclopean visual field map, in which is shown topological rendering of torus or dome surface, marked central fixation, shared field, unshared periphery, and vergence plane layers.

In certain implementations, the system can generate a moving dichoptic color-segregated stimulus in red and green color channels, controlling the spatial and temporal characteristics of the stimulus (e.g., motion speed, path, and location). A central yellow stimulus, as described above, provides a binocularly perceived fixation target that is simultaneously color-segregated when viewed through the filters. Also, peripheral moving stimuli are presented in coordinated red-green pairs to allow precise determination of whether the stimulus is detected by one eye, the other, or both.

The system records subject responses to the stimuli while also tracking real-time gaze position. The system computes the field location at which each monocular component is detected, and where both components are seen simultaneously (i.e., binocular fusion zone). For example, the system can record where in the field yellow, green, red, or “nothing” was perceived. By analyzing the transitions between these perceptions and their spatial locations (corrected for gaze angle and fixation depth), the algorithm builds a Cyclopean visual field map: Central fused field (yellow), Peripheral monocular fields (red/green), and Blind spots (no perception). These data are merged into a Cyclopean visual field map, in which each point is tagged as being: Perceived by the left eye only, perceived by the right eye only, and perceived by both eyes (binocular overlap). The result is a spatially explicit map of the shared binocular field and of the monocular-only peripheries. This software-controlled process enables precise quantification of the transition zones between monocular and binocular fields as a function of gaze angle and fixation depth, which is a key innovation of this invention. Using real time eye-tracking to verify that the subject maintains fixation on the central target. Throughout the test, the subject must maintain gaze on the central fixation marker. The eye-tracker may confirm that fixation is stable, for example, if fixation is lost, the moving stimulus path is paused or invalidated. This ensures that responses are mapped to the correct field locations.

In brief, by recording where the subject perceives yellow, red, green, or nothing, and combining this with real-time gaze and fixation depth data, the system constructs a Cyclopean visual field map: Central fused binocular field (yellow); Peripheral monocular fields (red or green); and Blind spots (no perception). This system enables such a map to be generated using a non-occlusive, binocular visual field test, providing new insights into real-world binocular vision.

In one aspect, disclosed is a binocular visual field assessment system that includes a display configured to present visual stimuli separately to each eye of a subject using a dichoptic delivery mechanism; an eye-tracking apparatus configured to detect gaze position and vergence angles of the subject in real time; a variable fixation platform configured to project stimuli at a plurality of fixation distances along the Z-axis; a control module operatively coupled to the display and eye-tracking apparatus; and a non-Euclidean computational processor.

The non-Euclidean computational processor is configured to: map vergence-dependent field boundaries using a spiral logarithmic encoding function; compute a Jacobian determinant from the vergence angle to quantify differential area growth; and generate a three-dimensional visual field map representative of a cyclopean percept derived from binocular integration.

In one aspect, disclosed is a method for performing binocular visual field assessment comprising the steps of: presenting a red-green dichoptic stimulus to a subject to isolate left-eye and right-eye input; capturing eye movement and vergence angle through a real-time eye-tracking mechanism; computing a spiral-logarithmic depth projection for each fixation plane; calculating a Jacobian area growth function to determine differential sensitivity at each depth plane; and rendering a cyclopean visual field map in three-dimensional space based on binocular fusion and vergence-dependent topological field distortion. The spiral-logarithmic depth projection is defined by the equation:

$$θ = \frac{1}{λ} log ( \frac{Z}{{Z}_{0}} )$$

wherein Z is the fixation distance, Z₀ is a reference distance, λ is a geometric constant, and θ is the vergence angle. Thee Jacobian determinant is computed as:

$$J = \frac{d A}{d Z} = π {θ}^{2}$$

to quantify perceived field expansion or compression as a function of fixation distance.

The method may further include the step of comparing the subject's cyclopean field map with a normative template to identify pathological deviations, including asymmetries or reduced peripheral boundaries.

In one aspect, the visual field map is generated as a toroidal surface or topological shell representing fused binocular input across shared and unshared visual zones. The output is used to quantify the boundary between central and peripheral vision and detect topological asymmetries in healthy or pathological conditions. The eye-tracking apparatus comprises a binocular high-speed infrared tracking unit synchronized to stimulus delivery and vergence calibration routines. The control module adjusts stimulus presentation dynamically based on real-time convergence angle, enabling kinetic perimetry with depth-based feedback.

The display is configured to deliver dichoptic stimuli using wavelength specific filters, polarized glasses, or split-screen LED presentation to independently stimulate each retina. The fixation distance can be varied using motorized adjustment of stimulus position along the anterior-posterior Z-axis, corresponding to near (≤50 cm), mid (75-100 cm), and far (≥150 cm) viewing distances.

The control module can adapt stimulus position and interocular disparity in real time based on captured vergence data to maintain alignment with the subject's fixation plane. The software outputs include a three-dimensional visual field map, a vergence angle distribution, a boundary detection index, and a topological deviation score from normative data.

The computational processor includes a machine-readable medium storing instruction that computes spiral projection and Jacobian transformations to generate a fused cyclopean field. The toroidal cyclopean map comprises continuous gradients of angular sensitivity and supports rendering of vergence-dependent field expansions across temporal and nasal hemifields. The data from the eye-tracking apparatus is sampled at ≥120 Hz and synchronized to stimulus presentation to ensure temporal precision in vergence-field mapping.

In certain embodiments, the system may further include a software module configured to classify visual field data into normative or pathological categories based on deviations from toroidal boundary metrics and spiral log vergence encoding patterns. The machine learning engine can be trained on normative cyclopean visual field maps and applied to detect asymmetries or field loss progression in patient test data. The repeated visual field tests may be time-stamped and compared across sessions to derive progression vectors of boundary displacement and field topology shifts. The output maps may be compatible with virtual or augmented reality rendering platforms and enable immersive visualization of field volume and peripheral blind zones. The software module may apply topological persistence methods and differential geometry analysis to quantify deviations in central vs. peripheral visual field encoding.

In certain embodiments, the data output from the Jacobian and spiral-log modules is integrated with clinical health record platforms or remote telemedicine dashboards for off-site review and diagnosis. A user interface can display spiral depth curves, Jacobian area metrics, fixation-dependent topologies, and clinical thresholds labeled by anatomical or functional region.

What is claimed is:

1. A binocular visual field assessment system comprising:
a processor and a memory;
a display device configured to render a screen presenting dichoptic, color-segregated visual stimuli to the eyes of a subject; and
an eye-tracking apparatus configured to capture eye tracking data in response to the visual stimuli at a plurality of fixation distances along a z-axis relative to the screen in an x-y plane,
wherein the processor is configured to processing eye tracking data corresponding to the plurality of fixation distances to generate a three-dimensional visual field map representative of a cyclopean perception derived from binocular integration.

2. The binocular visual field assessment system according to claim 1, wherein the eye tracking data comprises gaze position, vergence angles of the two eyes, and binocular field boundaries.

3. The binocular visual field assessment system according to claim 2, wherein dichoptic color segregated visual stimuli comprises:
a moving stimulus of a yellow color;
a fixation target configured to hold gaze, the fixation target is of a predefined color, wherein the yellow color moving stimulus is configured to isolate left-eye and right-eye input using a red and green anaglyph glasses to be worn by the subject, wherein the system is configured to move the moving stimulus across a visual field of the subject in a predetermined manner.

4. The binocular visual field assessment system according to claim 3, wherein the processing step for generating the three-dimensional visual field map comprises:
mapping vergence-dependent binocular field boundaries using a spiral logarithmic encoding function; and
computing a Jacobian determinant from the vergence angle to quantify differential area growth.

5. The binocular visual field assessment system according to claim 4, wherein the spiral logarithmic encoding function is defined by the equation:
$$θ = \frac{1}{λ} log ( \frac{Z}{{Z}_{0}} )$$
where Z is the fixation distance, Z₀ is a reference distance, λ is a geometric constant, and θ is the vergence angle,
wherein the Jacobian determinant is computed as:
$$J = \frac{d A}{d Z} = π {θ}^{2}$$
to quantify perceived field expansion or compression as a function of fixation distance.

6. The binocular visual field assessment system according to claim 4, wherein the binocular field boundaries comprises:
a shared binocular field, wherein both eyes perceiving the moving stimulus as yellow;
an unshared monocular filed, wherein an active eye of the two eyes percieves the moving stimulus as green or red; and
blind spots.

7. The binocular visual field assessment system according to claim 6, wherein blind spots are determined when the moving stimulus in the shared binocular field is perceved as red or green, and when the moving stimulas is pecieved to be disappeared.

8. A method for performing binocular visual field assessment comprising:
rendering a screen on a display by an interface module, upon execution by a processor, wherein the screen is configured to present a dichoptic color segregated visual stimuli to eyes of a subject;
determining eye tracking data, using an eye-tracking apparatus, in response to the dichoptic color segregated visual stimuli at a plurality of fixation distances between the screen and the eyes of the subject, wherein the screen is along a x-y plane and the plurality of fixation distances along a z-axis; and
processing the eye tracking data relative to the plurality of fixation distances to generate a three-dimensional visual field map representative of a cyclopean perception derived from binocular integration.

9. The method according to claim 8, wherein the eye tracking data comprises gaze position, vergence angles of the two eyes, and binocular field boundaries.

10. The method according to claim 9, wherein the method further comprises:
wearing a red and green anaglyph glasses by the subject, wherein the dichoptic color segregated visual stimuli comprises a moving stimulus and a fixation target, the fixation target is configured to hold gaze, the moving stimulus is of a yellow color and the fixation target is of a predefined color, wherein the yellow color moving stimulus is configured to isolate left-eye and right-eye input using the red and green anaglyph glasses; and
moving the moving stimulus across a visual field of the subject in a predetermined manner.

11. The method according to claim 10, wherein the processing step for generating the three-dimensional visual field map comprises:
mapping vergence-dependent binocular field boundaries using a spiral logarithmic encoding function; and
computing a Jacobian determinant from the vergence angle to quantify differential area growth.

12. The method according to claim 11, wherein the spiral logarithmic encoding function is defined by the equation:
$$θ = \frac{1}{λ} log ( \frac{Z}{{Z}_{0}} )$$
where Z is the fixation distance, Z₀ is a reference distance, λ is a geometric constant, and θ is the vergence angle,
wherein the Jacobian determinant is computed as:
$$J = \frac{d A}{d Z} = π {θ}^{2}$$
to quantify perceived field expansion or compression as a function of fixation distance.

13. The method according to claim 11, wherein the binocular field boundaries comprises:
shared binocular field, wherein both eyes perceiving the moving stimulus as yellow;
unshared monocular filed, wherein an active eye of the two eyes percieves the moving stimulus as green or red; and
blind spots.

14. The method according to claim 13, wherein blind spots are determined when the moving stimulus in the shared binocular field is perceived as red or green, and when the moving stimulas disappears to one or both eyes.

15. The method of claim 8, wherein the three-dimensional visual field map is generated as a toroidal surface or topological shell representing fused binocular input across shared and unshared visual zones.

16. The method of claim 15, wherein the toroidal cyclopean map comprises continuous gradients of angular sensitivity and supports rendering of vergence-dependent field expansions across temporal and nasal hemifields.

17. The method of claim 10, wherein the method further comprises:
adjusting stimulus presentation dynamically based on real-time convergence angle, enabling kinetic perimetry with depth-based feedback.

18. The method of claim 15, wherein the method further comprises
classifying visual field data into normative or pathological categories based on deviations from toroidal boundary metrics and spiral log vergence encoding patterns.
